(19) United States
(12) Patent Application Publication
(10) Pub. No.: US 2008/0200939 A1
(43) Pub. Date: Aug. 21, 2008

(54) LAPAROSCOPIC TOURNIQUET

(76) Inventors: Brian Maclean, Acton, MA (US); Todd Sloan, Medway, MA (US)

Correspondence Address: FAY KAPLUN & MARCIN, LLP, 150 BROADWAY, SUITE 702, NEW YORK, NY, 10038, US

(21) Appl. No.: 12/019,214

(22) Filed: Jan. 24, 2008

Related U.S. Application Data
(60) Provisional application No. 60/890,651, filed on Feb. 20, 2007.

(51) Int. Cl. A61B 17/08

(52) U.S. Cl. 606/158

(57) ABSTRACT

A method for necrosing target tissue within a hollow organ, comprises looping a cinching device around an outer surface of the hollow organ and coupling ends of the cinching device to one another in combination with tightening the cinching device to compress tissue of the hollow organ to occlude at least one artery supplying blood to the hollow organ and maintaining the cinching device around the hollow organ until a period of time selected to necrose the target tissue has elapsed.

### PRIORITY CLAIM

This application claims the priority to the U.S. Provisional Application Ser. No. 60/890,651, entitled “Laparoscopic Tourniquet,” filed Feb. 20, 2007. The specification of the above-identified application is incorporated herewith by reference.

### BACKGROUND INFORMATION

Uterine fibroids are a common condition causing abnormal menstrual bleeding. Conventional treatments include drug therapies that are generally better suited for less advanced cases, and hysterectomies, or surgical removal of the uterus for more serious cases. However, less invasive alternative procedures are often preferable as they typically entail fewer side effects, shorter hospital stays, and reduced discomfort.

For example, diverse methods of ablating tissue have been employed to endoscopically remove abnormal growths from inner walls of the uterus. These procedures are local in nature, with each fibroids being treated individually. In these procedures, a hysteroscope or similar device is maneuvered into position to locate a first target portion of tissue and to carry out the treatment of this first portion of target tissue. The hysteroscope is then repositioned for the subsequent portion of target tissue to be treated and the process is repeated for each additional portion of target tissue. These repeated treatments are time consuming and the need to treat each target portion individually involves the risk that one or more target portions of tissue will be overlooked.

Alternatively, fibroids have been treated by occluding blood supply to the target tissue. Such procedures generally involve the deployment of a clamp or similar device to occlude blood vessels supplying the target tissue. Accessing the appropriate blood vessels may be difficult and the clamps used to apply compression may damage the blood vessels as well as the surrounding tissue. In addition, such occlusion procedures carry the risk of infection or other complications if clamps are not properly placed and they can be painful.

Global treatment procedures which treat all fibroids within the uterus at the same time involve the interruption of the blood supply to the entire uterus for a time period selected to necrose all the fibroids without permanently damaging non-targeted tissue. Uterine artery embolization is one conventional global treatment that involves filling one or both uterine arteries intravascularly to block the flow of blood. However, this treatment is costly because it is carried out by an interventional radiologist under fluoroscopy.

For example, as shown in FIG. 2, a clamping device 112 generally shaped like a pair of tongs is placed in the vagina so that the arms 113 clamp on the cervix 108 and force it to close. The clamping force causes the tissue of the vagina 120 to move proximally to the cervix 108 and apply pressure on the uterine arteries 102, 104 that are located about one centimeter proximal to the opening of the cervix 108. The pressure from the clamping device 112 restricts the flow of blood to the uterus and thus to the target uterine fibroids. After a desired period of time, the clamping device 112 is removed to restore blood flow to the uterus.

### SUMMARY OF INVENTION

The present invention is directed to a method for necrosing target tissue within a hollow organ, comprises looping a cinching device around an outer surface of the hollow organ and coupling ends of the cinching device to one another in combination with tightening the cinching device to compress tissue of the hollow organ to occlude at least one artery supplying blood to the hollow organ and maintaining the cinching device around the hollow organ until a period of time selected to necrose the target tissue has elapsed.

### BRIEF DESCRIPTION OF DRAWINGS

FIG. 1 is a sectional view showing a uterus with several uterine fibroids and the location of the uterine arteries;

FIG. 2 is a schematic sectional view of the uterus showing a conventional arterial occlusion device; and

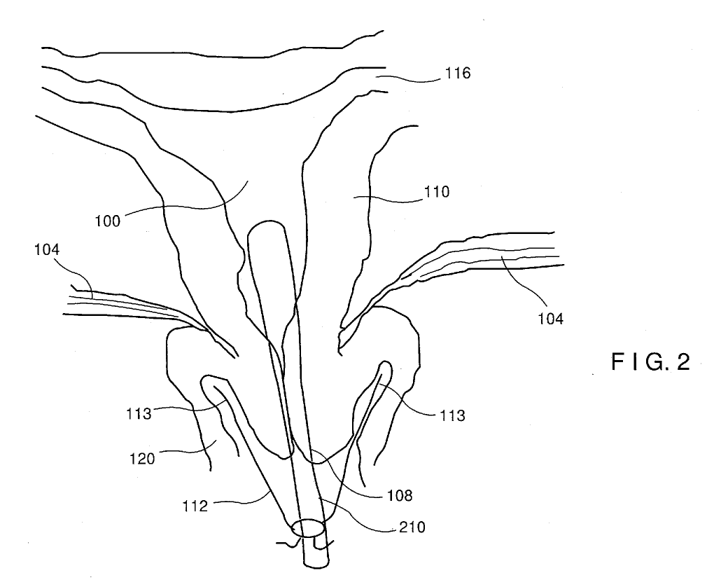

FIG. 3 is a sectional view of the uterus showing an occlusion tourniquet according to an embodiment of the present invention.

### DETAILED DESCRIPTION

The present invention may be further understood with reference to the following description and to the appended drawings, wherein like elements are referred to with the same reference numerals. The present invention relates to devices for reducing or stopping the flow of blood to fibroids or other target tissue within an organ. In particular, the present invention relates to devices for temporarily occluding the flow of blood from the uterine arteries to the uterus and/or blood vessels within the uterus itself.

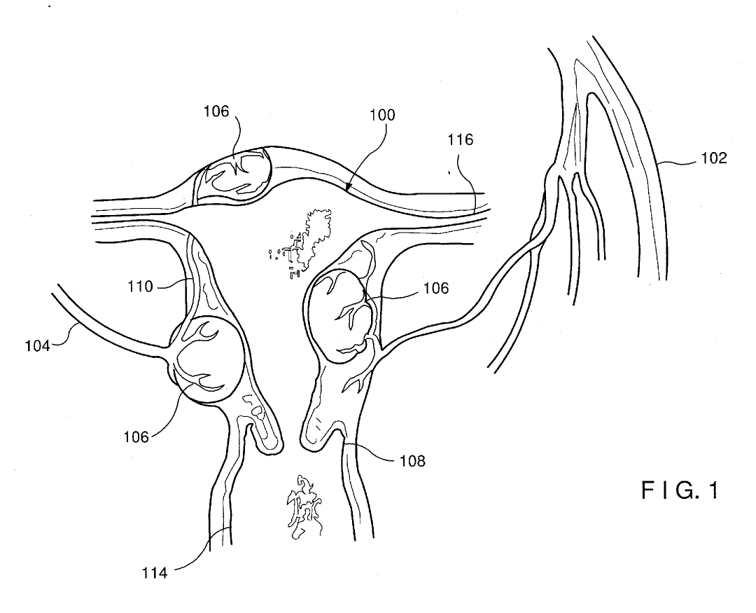

The exemplary embodiments of the present invention provide a method and a system for treating target tissue which is less complex and which reduces recovery time and adverse side effects. According to embodiments of the invention, uterine fibroids are necrosed by placing a tie around the uterus and tightening it to temporarily cinch the uterus and block the flow of blood thereto. After an amount of time sufficient to ensure necrosis of the fibroids has elapsed, the tie is released and/or removed. As would be understood by those skilled in the art, the time period for which blood flow is interrupted is selected to be shorter than a time at which the non-targeted tissue of the uterus would be damaged. In addition, those skilled in the art will understand that as an alternative to releasing or removing the tie, the tie may be formed of a material which will be absorbed into the body after a selected period of time so that the force applied thereby will permit blood flow to the uterus after the end of the time period during which it is desired to interrupt the blood flow. The time period required to necrose uterine fibroids has been shown to be approximately six hours which is a time period short enough to prevent or minimize damage to non-targeted tissues. As shown in FIG. 1, the uterus 100 receives its principal blood supply from two uterine arteries 102, 104. One or more uterine fibroids 106 may form in the uterine walls 110 at various locations, that may be at different distances from the uterine arteries 102, 104.

According to an exemplary embodiment of the present invention, a standard laparoscopic procedure is used to approach the uterus from within the abdomen and to thread a tying device such as a tourniquet around the uterus. As would be understood by those skilled in the art, the uterus is located beneath the bladder and is movable within its surrounding tissue so that it can be manipulated to place the tourniquet in a desired position. At the physician's discretion, the tourniquet may be placed adjacent to the uterine arteries, distal or proximal thereto, depending on the location of the fibroids to be treated. The closer the tourniquet is placed to the uterine arteries, the more likely it is that all the fibroids within the organ will necrose from lack of blood. The placement of the tourniquet can therefore be optimized according to the location of the fibroids to achieve the complete necrosis of all the fibroids in the uterus.

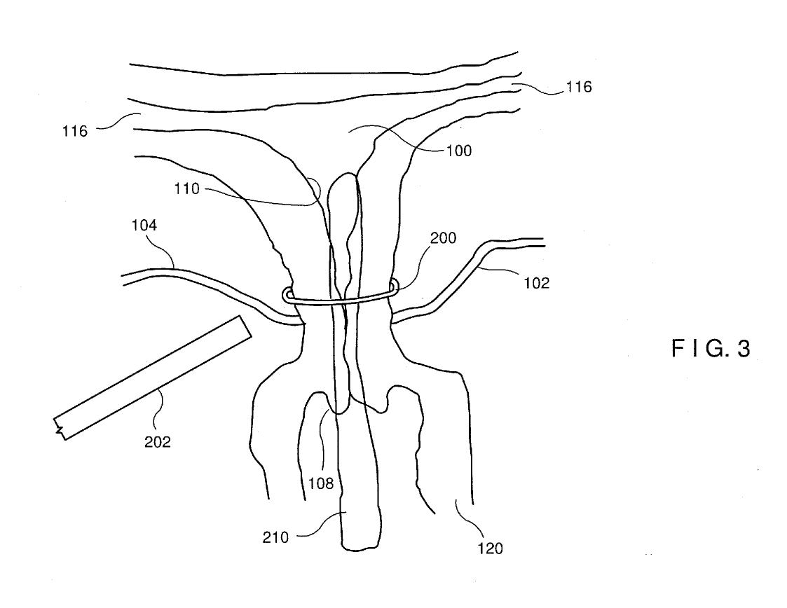

FIG. 3 shows an exemplary embodiment of a uterine tourniquet according to the present invention. The exemplary embodiment comprises a cinching device 200 which may, for example, be formed as a tie or tourniquet that is placed around the uterus 100 under direct visualization with laparoscopic techniques as would be understood by those skilled in the art. The cinching device 200 may comprise a suture loop which is tightened around a portion of the uterus 100 and may, for example, be formed of a biodegradable material designed to dissolve after residing within the body for a defined period of time selected to achieve necrosis of the fibroids. Alternatively, the cinching device 200 may be surgically removed after the procedure has been completed. As would be understood by those skilled in the art, a cinching device (not shown) according to a still further embodiment of the invention, may be a Capio-like device having a predefined curvature with a tube (e.g., Nitinol) which, when activated would extend to a position in which it is curved around the uterus to feed the suture therearound and which, when retracted, would leave the suture in place around the uterus. The physician may then tighten the suture to restrict blood flow to the uterus.

The cinching device 200 may comprise different materials and methods of construction. For example, a narrow band of mesh material may be used instead of a suture, or any other medically suitable component that may be employed which is biocompatible, flexible enough to be wrapped and maintained in position around the uterus 100 and which has the strength required to maintain a compressive force on the uterus 100 sufficient to cut off the blood supply thereto.

In the exemplary embodiment shown, the cinching device 200 is delivered below the level of the fallopian tubes 116, using a steerable catheter type device 202 through a trocar (not shown) as would be understood by those skilled in the art. Other conventional delivery methods may be used to place the cinching device 200 in proximity to the uterus 100 from within the abdominal cavity The exemplary steerable catheter may, for example, be a simple uni-directional deflectable device with the deflection effected via one or more pullwires or other conventional means. Once the cinching device 200 has been placed in a desired position around a selected portion of the uterus 100, the ends 202 thereof are grasped, for example, with a forceps or other known grasping device (not shown in the drawings), and pulled out through a single trocar. The ends 202 of the cinching device 200 are then tied into a knot that is pushed down to tighten around the neck of the cervix 108, and apply mechanical pressure to the wall of the uterus 110. Those skilled in the art will understand that, as an alternative to tying a knot in the suture, the device 200 may employ loop attachments, any dissolving and/or snap attachments, tongue and groove attachments, or known tightening mechanisms such as a ratchet lock with a series of barbed projections which may be pulled through the lock in a locking direction but which extend to prevent loosening. Furthermore, the ends of the suture may alternatively be thermally formed, microultrasonicly welded to one another.

According to the exemplary embodiment of the invention, multiple knots may be tied and pushed down to tighten around the cervix 108 more securely. The knot or knots apply mechanical pressure on the uterine walls 110 and compress the uterine arteries 102, 104 restricting or completely occluding the flow of blood therethrough. As described above, the cinching device 200 may be formed of a biodegradable material selected to maintain the compressive force on the uterus 100 for a selected period of time before dissolving. Alternatively, the compressive force may be removed through a follow up procedure to remove the cinching device 200.

In another exemplary embodiment of the present invention, a solid structure 210 which may, for example, be a sound, is inserted through the cervix 108 into the uterus 100 to enhance and make more uniform the compression of the uterine walls and, consequently, of the uterine arteries 102, 104 by the cinching device 200. The position of the solid structure 210 within the uterus 100 is preferably selected to facilitate occlusion of the blood supply to specific fibroids 106 located within the organ by providing a rigid backing against which the tissue of the uterus is compressed by the device 200. For example, the solid structure 210 may be positioned to occlude either branches of the uterine arteries identified as supplying the fibroids or to occlude the uterine arteries themselves.

The cinching device 200 is preferably placed along the uterus 100 at a location selected to maximize the effectiveness of the occlusion in treating the fibroids. For example, the location of the cinching device 200 is preferably below a position of the fallopian tubes but above a position of one or both of the uterine arteries. In this context, below refers to a position toward the cervix 108, and above refers to a position toward the fallopian tubes 116. The location of the cinching device 200 is also preferably selected based on a location of one or more of the fibroids present to occlude a supply of blood to the uterine fibroids possibly in conjunction with the position of a solid structure 210 as described above.

The present invention has been described with reference to specific exemplary embodiments. Those skilled in the art will understand that changes may be made in details, particularly in matters of shape, size, material and arrangement of parts.

Accordingly, various modifications and changes may be made to the embodiments without departing from the teachings of the invention. The specification and drawings are, therefore, to be regarded in an illustrative rather than a restrictive sense.

What is claimed is:

1. A method for necrosing target tissue within a hollow organ, comprising:
looping a cinching device around an outer surface of the hollow organ;
coupling ends of the cinching device to one another; and
tightening the cinching device to compress tissue of the hollow organ to occlude at least one artery supplying blood to the hollow organ and maintaining the cinching device around the hollow organ until a period of time selected to necrose the target tissue has elapsed.

2. The method of claim 1, wherein the hollow organ is the uterus and the target tissue comprises one or more fibroids.

3. The method of claim 2, further comprising selecting a placement location for the cinching device along the uterus between fallopian tubes and a uterine artery supplying the target tissue.

4. The method of claim 1, further comprising inserting into the hollow organ a substantially axially incompressible member against which the hollow organ is to be compressed by the cinching device.

5. The method of claim 4, wherein the hollow organ is the uterus and the target tissue comprises one or more fibroids and wherein the substantially axially incompressible member comprises a sound.

6. The method of claim 1, wherein the cinching device comprises a length of suture, further comprising the step of coupling ends of the length of suture to one another to maintain a desired compression on the hollow organ.

7. The method of claim 6, wherein the ends of the length of suture are coupled to one another by one of knotting, adhesive and a mechanical coupling.

8. The method of claim 1, wherein the cinching device is looped around the hollow organ using an insertion system comprising a trocar and a steerable catheter.

9. The method of claim 6, wherein the length of suture is formed of a biodegradable material selected to degrade and release the compression of the hollow organ after the period of time selected to necrose the target tissue has elapsed.

10. The method of claim 1, further comprising performing a procedure to remove the cinching apparatus after the period of time selected to necrose the target tissue has elapsed.
